(19) United States
(12) Patent Application Publication
(10) Pub. No.: US 2012/0312726 A1
(43) Pub. Date: Dec. 13, 2012

(54) METHOD AND DEVICE FOR CHECKING THE DELIVERY PERFORMANCE OF AT LEAST ONE DELIVERY MEANS OF A DEVICE FOR EXTRACORPOREAL BLOOD TREATMENT

(76) Inventor: Alfred Gagel, Litzendorf (DE)

(21) Appl. No.: 13/493,310

(22) Filed: Jun. 11, 2012

Related U.S. Application Data
(60) Provisional application No. 61/495,054, filed on Jun. 9, 2011.

(30) Foreign Application Priority Data
Jun. 9, 2011 (DE) 10 2011 106 113.8

(51) Int. Cl. G01F 1/34; G06F 19/00; A61M 1/14

(52) U.S. Cl. 210/143; 73/861.42; 702/47

(57) ABSTRACT

Method and device for checking on the delivery performance of at least one first and/or second delivery means of a device for extracorporeal blood treatment by measuring the pressure in a closed container filled at least partially with air by a first pressure measurement, delivering a first liquid volume into the container with the first delivery means, measuring the pressure in the container by a second pressure measurement after delivering the first liquid volume, delivering a second liquid volume out of the container with the second delivery means, measuring the pressure in the container by a third pressure measurement after delivering the second liquid volume out of the container, and evaluating at least the measured values of the first pressure measurement and the third pressure measurement as a criterion for the deviation in the delivery performance.

The invention relates to a device in a method for checking on the delivery performance of at least one delivery means for medical fluids on devices for extracorporeal blood treatment as well as a blood treatment device.

There are various known types of blood treatment devices. Known blood treatment devices include those for hemodialysis, hemofiltration and hemodiafiltration, for example. During an extracorporeal blood treatment, blood flows through a blood treatment unit in an extracorporeal blood circulation. Of the devices for hemodialysis, hemofiltration and hemodiafiltration, the blood treatment unit is a dialyzer or filter, which, in simplified terms, is separated by a semipermeable membrane into a blood chamber and a dialysis fluid chamber. During a blood treatment by hemodialysis or hemodiafiltration, blood flows through the blood chamber while dialysis fluid flows through the dialysis fluid chamber.

Dialysis fluid may be supplied through a dialysis fluid system integrated into the device for extracorporeal blood treatment. Clean water, e.g., from reverse osmosis, may be supplied to the dialysis fluid system after first being degassed and mixed with liquid concentrates to prepare fresh dialysis fluid. Mixing may be done by adding liquid concentrates to the clean water line at separate addition points, for example, and then mixing thoroughly in a mixing chamber or by adding the liquid concentrates directly to a mixing chamber through separate feed points. Fresh dialysis fluid flows through a balancing system first and then is sent through the dialysis chamber of the dialyzer. Fresh dialysis fluid then becomes loaded with water and ingredients from the blood and thereby becomes spent dialysis fluid. After leaving the dialyzer, the spent dialysis fluid passes through the balancing system. Any difference between the volume of the fresh dialysis fluid and the spent dialysis fluid is determined there.

The mixing chamber has incoming fluid lines and outgoing fluid lines. The mixing chamber may receive partially premixed mixture from degassed clean water and liquid concentrates. Complete mixing is done in the mixing chamber. Fresh dialysis fluid is removed from the mixing chamber through a dialysis fluid line. Clean water, liquid concentrates and dialysis fluid are delivered in the lines by delivery means, for example, by pumps. The clean water is degassed by creating a vacuum by means of a degassing pump in the clean water line upstream from the mixing chamber. Liquid concentrates are delivered by dosing pumps upstream from the mixing chamber. Dialysis fluid is delivered by a dialysis fluid pump in the dialysis fluid line. Additional pumps, for example, the flow pump in the dialysis fluid line and an ultrafiltration pump may be in fluid connection with the dialysis fluid line up-stream from dialyzer and with the dialysis fluid line downstream from the dialyzer.

The dialysis fluid in blood treatment machines can be prepared by volumetric mixing. Volumetric mixing is understood to mean that at least one liquid is dosed according to the volume. For example, clean water and at least one liquid concentrate may be dosed volumetrically and mixed according to a given recipe to yield fresh dialysis fluid.

In volumetric dosing, the accuracy of the delivery performance of the delivery means is of crucial importance. Faulty dosing leads to a faulty dialysis fluid composition. The delivery performance of a delivery means may vary over time. Causes of such changes in delivery performance of delivery means may include, for example, leakage, swelling of materials, deposits inside the delivery means and/or the lines and wear. Although leakage can be detected easily and reliably with an automatic integrity test, for example, a pressure holding test, the other causes which may lead to a larger or smaller delivery performance cannot be easily be detected automatically.

In order not to endanger patient safety, faulty dosing must be prevented. Therefore the delivery means must be designed to be so sturdy that an operational change in the delivery performance during its lifetime is as minor as possible. The delivery performance of delivery means may nevertheless change over time.

The delivery performance is checked at regular maintenance intervals and corrected as needed. The delivery performance can be determined by measuring the volume in liters. Such work is performed by a qualified service technician.

The more often the delivery means of the blood treatment device are checked, the more reliably the blood treatment device can function. There is a demand for a regular automatic checking of the delivery performance. There is a demand for automatic detection of changes in the delivery performance of a delivery means, for example, as part of a regular automatic function test.

One object of the invention is to improve upon a generic blood treatment device, such that the delivery performance of at least one first or one second delivery means can be checked automatically without requiring the use of a qualified service technician or a user.

Another object of the invention is to improve upon a generic blood treatment device, so that even detecting a deviation in the delivery performance of at least one first or one second delivery means from the target value range triggers an alarm without necessarily ascertaining which of the two delivery means has a deviation in the delivery performance or whether both delivery means have a deviation in the delivery performance.

Another object includes not only detecting a deviation in the delivery performance of at least one first or one second delivery means from the target value range but also includes another step for ascertaining which of the two delivery means has a deviation in the delivery performance or whether both delivery means have a deviation in the delivery performance.

Another object of the present invention is to improve upon the user friendliness of a generic blood treatment device, so that the user, for example, the treating physician or a dialysis nurse is not burdened with having to check on and correct the delivery performance of delivery means, and therefore there cannot be any downtime of the blood treatment machine while a qualified service technician is servicing the machine. Therefore there should be automatic correction of the delivery performance of the at least one first or second delivery means when there is a deviation in its delivery performance from the target value range.

Another object of the present invention is to increase the reliability of the blood treatment device.

Another object of the present invention is to increase the safety of the blood treatment device. The more accurately the delivery means of the blood treatment device can deliver the fluid, the more reliably the blood treatment device can operate.

The solution to these problems is achieved according to the invention with the features of Independent Patent claims 1, 12, 26, 27 and 28. Advantageous embodiments are the subject matter of the dependent claims. The advantages of the inventive method according to claim 1 can be achieved undiminished with the device according to claim 12 and a device for extracorporeal blood treatment according to claim 26. In certain embodiments, the advantages of the inventive method can be achieved undiminished with the computer program product according to claim 27 and the computer program according to claim 28.

According to the teaching of the invention, these problems are solved by measuring the pressure in a closed container filled at least partially with air by means of a first pressure measurement, delivering a first liquid volume with a first delivery means into the container, measuring the pressure in the container with a second pressure measurement after delivering the first liquid volume into the container, delivering a second liquid volume with second delivery means out of the container, measuring the pressure in the container by means of a third pressure measurement after delivering the second liquid volume out of the container and analyzing at least the measured values of the first pressure measurement and the third pressure measurement as a criterion for the deviation in the delivery performance of the second delivery means from the delivery performance of the first delivery means. By delivering the first liquid volume, the air volume in the container is compressed and the pressure increases from the measured value of the first pressure measurement to the measured value of the second pressure measurement. By delivering the second liquid volume, the compressed air volume is depressurized again and the pressure in the container drops from the measured value of the second pressure measurement to the measured value of the third pressure measurement. This sequence of delivery operations, namely delivery into the container first and then delivery out of the container, is defined as the first delivery sequence.

The teaching of the present invention also includes the special case when there is no air in the container but the elasticity of the container and optionally the elasticity of the lines are designed to be great enough to receive the liquid volume delivered. This special case may be applied when very small delivery volumes are to be measured with a high precision. Starting from this special case by providing a suitable air volume in the container, the compliance can be adapted to the container volumes and pressures prevailing in the application case.

Naturally it is also possible to proceed in the reverse order according to the invention by achieving the objects according to the teaching of the invention by measuring the pressure in a closed container filled at least partially with air and at least partially with liquid by means of a first pressure measurement, delivering a first liquid volume by means of a first delivery means out of the container, measuring the pressure in the container by means of a second pressure measurement after delivering the first liquid volume out of the container, delivering a second liquid volume by means of a second delivery means into the closed container, measuring the pressure in the container by means of a third pressure measurement after delivering the second liquid volume into the container and analyzing at least the measured values of the first pressure measurement and of the third pressure measurement as a measure of the deviation in the delivery performance of the second delivery means from the delivery performance of the first delivery means. In this order, a sufficient liquid volume in the container is of course necessary before delivering a first liquid volume out of the container by means of a first delivery means. By delivering the first liquid volume, the air volume in the container is depressurized and the pressure drops from the measured value of the first pressure measurement to the measured value of the second pressure measurement. By delivering the second liquid volume into the container, the depressurized air volume is compressed again and the pressure in the container rises from the measured value of the second pressure measurement to the measured value of the third pressure measurement. This sequence of delivery processes, namely first delivery out of the container and then delivery into the container, is defined as the second delivery sequence.

In addition, in many embodiments, checking the delivery performance may also include correcting the delivery performance. Correcting may be understood to refer to eliminating a faulty deviation. Correcting may in particular be understood to refer to increasing or decreasing the delivery performance until reaching a target value. The delivery performance may be corrected by a control intervention measure on the delivery means or on the electric drive of the delivery means.

According to the invention, a deviation in the delivery performance of the first delivery means from the delivery performance of the second delivery means may be determined as a criterion for checking the delivery performance of the first delivery means and/or of the second delivery means or may be determined merely as a criterion of a deviation in the delivery performance of the first delivery means from the delivery performance of the second delivery means. A criterion of a deviation in the delivery performance of the first delivery means from the delivery performance of the second delivery means may be, for example, a measured pressure, a calculated pressure difference from measured pressures or a calculated volume difference from the first and second liquid volumes delivered according to the invention.

In certain embodiments, a measure of a deviation in the delivery performance may be obtained by comparing at least one measured pressure with a lower limit value and/or an upper limit value. On falling below the lower limit value or exceeding the upper limit value, an unallowed deviation in the delivery performance of the at least one first and/or second delivery means may be deduced. For example, at least the measured values of the first pressure measurement and the third pressure measurement may be compared.

In other embodiments, the analysis of at least the measured values of the first pressure measurement and the third pressure measurement may include comparing the measured value of the third pressure measurement with an upper limit value or a lower limit value.

The upper limit value and the lower limit value may be defined, for example, as allowed up and/or down deviations with respect to the measured value of the first pressure measurement or with respect to a predetermined pressure target value.

At first it is impossible to determine, merely on the basis of the analysis of at least one of the measured values of the first pressure measurement and the third pressure measurement, whether the first or second delivery means or even both delivery means have a defective delivery performance and how much the deviation amounts to. However, it is possible to ascertain whether the delivery performance of the second delivery means deviates inadmissibly from the delivery performance of the first delivery means or the delivery performance of the first delivery means deviates inadmissibly from the delivery performance of the second delivery means.

In a first error case, it is possible to ascertain by using the first delivery sequence that the delivery performance of the second delivery means inadmissibly exceeds the delivery performance of the first delivery means when the third pressure measurement falls below the lower pressure limit value. In a second error case, it is possible to ascertain that the delivery performance of the second delivery means is inadmissibly lower than the delivery performance of the first delivery means when the third pressure measurement exceeds the upper pressure limit value.

When using the second delivery sequence, it is possible to ascertain in a first error case that the delivery performance of the second delivery means inadmissibly exceeds the delivery performance of the first delivery means when the third pressure measurement exceeds the upper pressure limit value. In a second error case, it is possible to ascertain that the delivery performance of the second delivery means inadmissibly falls below the delivery performance of the first delivery means when the third pressure measurement falls below the lower pressure limit value.

In the simplest case, the first delivery means and the second delivery means have the same design and therefore have the same nominal delivery performance. In other cases, the first delivery means and the second delivery means may have different nominal delivery performances. The establishment of the lower limit value and the upper limit value defines a tolerance range of the pressure in the container merely for taking into account the admissible deviations in the delivery performance of the two delivery means from the nominal delivery performance.

The deviations in the delivery performance of the two delivery means may be due to the design. Known differences in the two delivery means may occur, for example, due to different nominal delivery performances when the first delivery means and the second delivery means are not of the same design. The establishment of the lower limit value and the upper limit value defines a tolerance range of the allowed deviation. This tolerance range may take into account an admissible deviation and the delivery performances of the two delivery means from their nominal delivery performances in the case of delivery means that are not of the same design. Furthermore, the tolerance range may take into account differences due to different design parameters of the two delivery means. One example of different design parameters of the two delivery means is different stroke volumes with two diaphragm pumps.

In other embodiments, a pressure difference, for example, a calculated pressure difference, may be determined from the measured value of the first pressure measurement and the measured value of the third pressure measurement as a criterion for the deviation in the delivery performance. The pressure difference may be compared with a lower limit value and/or an upper limit value. If the value is below the lower limit value or if it exceeds the upper limit value, an inadmissible deviation in the delivery performance of the at least one and/or the second delivery means may be concluded.

In many embodiments instead of the absolute pressure, the relative pressure in the container can be measured. This is true, for example, for embodiments which provide an analysis of the measured values of the first pressure measurement and of the third pressure measurement and a comparison of the measured value of the third pressure measurement with a lower limit value and/or an upper limit value. This is also true, for example, of other embodiments, which include comparing the pressure difference with an upper limit value and/or a lower limit value.

One criterion of a deviation in the delivery performance may be a volume difference in other embodiments. The volume difference may be compared with a lower limit value and/or an upper limit value. If the value is below the lower limit value or exceeds the upper limit value, an inadmissible deviation in the delivery performance of the first and/or the second delivery means may be deduced. Instead of the comparison of the third measured pressure value with a lower pressure limit value and an upper pressure limit value, a volume difference may be calculated from the measured pressure values on the basis of the Boyle-Mariotte law. The delivery volume difference may be positive or negative. For such embodiments, it is necessary to measure the absolute pressure in the container. An absolute pressure gauge is used to measure the pressure.

The following equation holds for the change in state in the container, based on the delivery of the first liquid volume:

P₀·V_{vessel,air,0}=P₁·(V_{vessel,air,0}−V_{pump,1}) (1)

For the change in state in the container based on the delivery of the second liquid volume, it holds that:

P₀·V_{vessel,air,0}=P₂·(V_{vessel,air,0}−V_{pump,1}+V_{pump,2}) (2)

In equations (1) and (2), P₀ denotes the absolute pressure in the container before delivery of the first liquid volume, V_{vessel,air,0} denotes the initial air volume in the container, V_{pump,1} denotes the first liquid volume after delivery with a first delivery means, V_{pump,2} denotes the second liquid volume after delivery with a second delivery means, P₁ denotes the absolute pressure in the container in the mixing chamber after delivery of the first liquid volume and P₂ denotes the absolute pressure in the mixing chamber after delivery of the second liquid volume.

Based on the calculated pressure difference according to equation (3), a delivery volume error can be deduced according to equation (4),

ΔP=P₂−P₀ (3)

ΔV=V_{pump,1}−V_{pump,2} (4).

With the help of the definition of compliance according to equation (5), the delivery volume error can be calculated according to equation (6).

$$\begin{matrix}C = \frac{Δ V}{Δ P} = \frac{{V}_{vessel , air , 0}}{{P}_{0}} & ( 5 ) \\ Δ V = C \cdot ( {P}_{2} - {P}_{0} ) & ( 6 )\end{matrix}$$

The compliance may be known as a system parameter. The initial air volume in the container can be determined by a filling level measurement. The pressures can be measured by an absolute pressure sensor. If no absolute pressure gauge is present, the absolute pressure may be determined automatically using a method and a device such as those described by the present applicant in another patent application with the title “Method and Device for Determining at Least One Operating Parameter of a Device for Extracorporeal Blood Treatment as a Function of the Absolute Pressure, Device for Extracorporeal Blood Treatment” (internal application no. 10/45-d01 DE), which has the same filing date at the present patent application. Reference is thus made to the full content of the aforementioned patent application.

The delivery volume error may be compared with an upper volume error limit value and a lower volume error limit value. Alternatively, it is also possible to compare only the delivery volume error with only a volume error limit value. The volume error limit values may be predetermined as up and/or down deviations based on a target value of at least one delivery volume. The up and/or down deviations may be based on the target value of the delivery volume of at least the first or the second delivery means.

In a first error case, when using the first delivery sequence and with specification of an upper limit value and a lower limit value each defined as a positive and as a negative deviation from the target value of the delivery volume of the first delivery means, it is possible to ascertain that that the delivery volume of the second delivery means inadmissibly exceeds the delivery volume of the first delivery means when the volume error according to equation (6) is below the lower volume error limit value. In a second error case, it is possible to ascertain that the delivery of the second delivery means inadmissibly falls below the delivery volume of the first delivery means when the delivery volume difference exceeds the upper volume error limit value. In the first and second error cases, it is possible to ascertain that at least the first or the second delivery means or both delivery means have a defective delivery performance. However, it cannot be ascertained at first which of the delivery means has the defective delivery performance, but this is adequate at first for error recognition.

By analogy with that, when using the second delivery sequence in a first error case it is possible to ascertain that the delivery volume of the second delivery means inadmissibly exceeds the delivery volume of the first delivery means when the volume error according to equation (6) exceeds the upper volume error limit value. In a second error case, it is possible to ascertain that the delivery volume of the second delivery means inadmissibly falls below the delivery volume of the first delivery means when the delivery volume difference falls below the lower volume error limit value.

The delivery of a first liquid volume or a second liquid volume may be discontinuous or continuous. Discontinuous delivery may be understood to refer to delivery by repeated pump strokes, such that the delivery occurs continuously during a single pump stroke.

Examples of pumps which deliver discontinuously include diaphragm pumps and piston pumps. The delivery performance with these pumps which may have a constant stroke volume is adjusted through the number of strokes per unit of time. A certain delivery volume is delivered by a certain number of delivery strokes.

Another example of a discontinuously delivering pump is a pump having a stepping motor which operates in steps, i.e., discontinuously. The delivery performance with such a pump may be set on the stepping motor, for example, by means of a control and computation unit while stipulating a step angle per delivery step and a number of steps per unit of time. A certain delivery volume may be set on the stepping motor with such a pump by means of a control and computation unit, for example, by stipulating a step angle per delivery step and a number of steps. However, for precise delivery, it is necessary for the pump to operate occlusively, so there cannot be any return flow.

Continuous delivery of a certain delivery volume with a gear pump may be set on a continuously rotating drive motor, for example, a brushless d.c. motor by means of a control and computation unit, for example, by stipulating a rotational speed and a delivery time.

An exemplary embodiment of the invention is explained in greater detail below with reference to the figures. Additional details and advantages of the invention are described in greater detail on the basis of the exemplary embodiment shown in the figures. The inventive method and the inventive device are described on the example of a blood treatment device, which is embodied as a hemodialysis device. However, the inventive method may also be used in the same way with other blood treatment devices, for example, a hemodiafiltration device.

The drawings show:

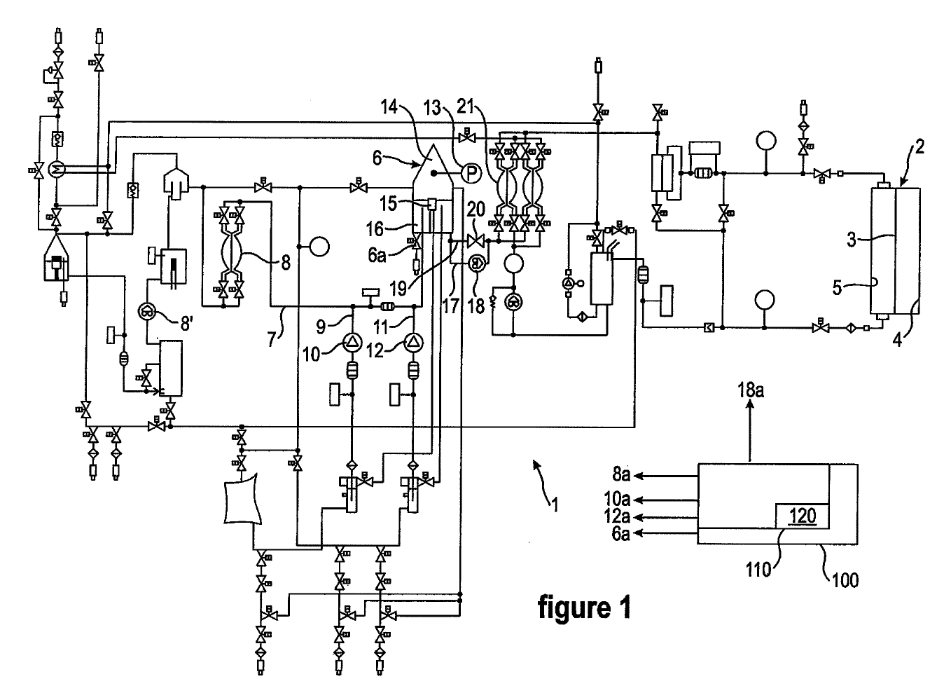

FIG. 1 a flowchart of the dialysate system of a blood treatment device designed as a hemodialysis device and having a mixing chamber;

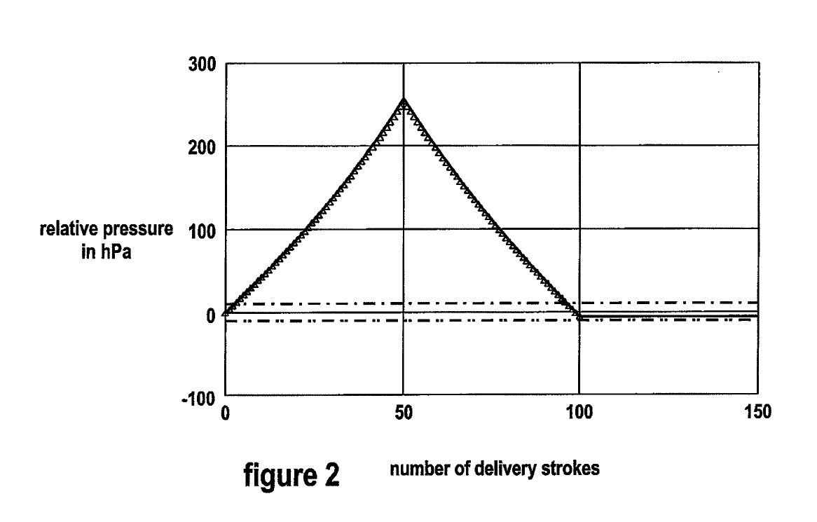

FIG. 2 a graphic plot of the changes in pressure in the mixing chamber of the dialysis fluid system from FIG. 1 in performing the inventive method with two delivery means with normal delivery performance;

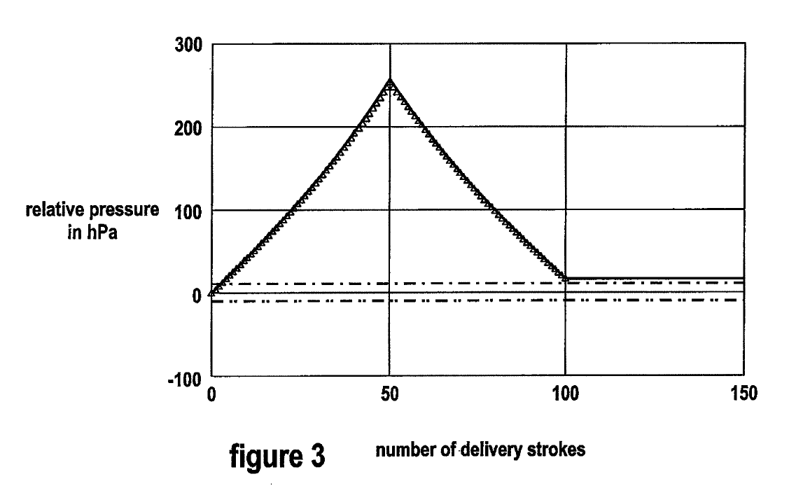

FIG. 3 shows a graphic plot of the changes in pressure in the mixing chamber of the dialysis fluid system from FIG. 1 in performing the inventive method with two delivery means with faulty delivery performance of at least the first or the second delivery means and exceeding the upper limit value for the relative pressure;

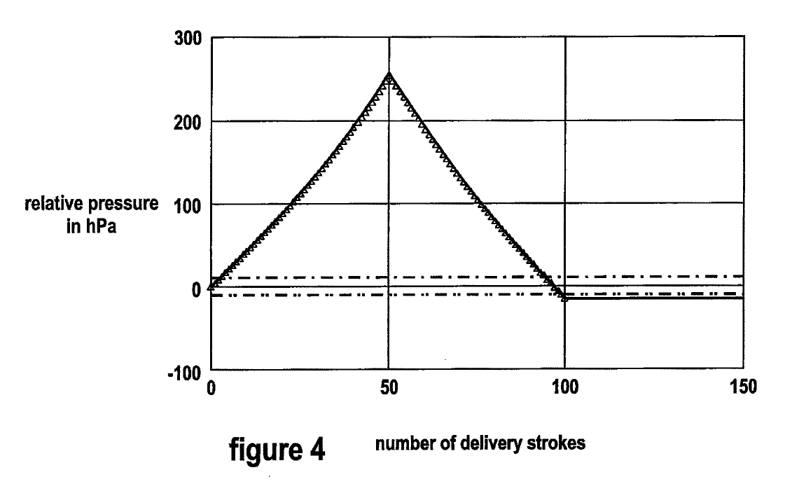

FIG. 4 a graphic plot of the pressure conditions of the relative pressure in the mixing chamber of the dialysis fluid system according to FIG. 1 in performing the inventive method with two delivery means having defective delivery performance of at least the first or the second delivery means and falling below the lower limit value for the relative pressure.

In a simplified schematic diagram, FIG. 1 shows the essential components of the dialysis fluid system 1 of a blood treatment device designed as a hemodialysis device. In the present exemplary embodiment, the blood treatment device is a hemodialysis device, having a dialyzer 2, which is separated schematically by a semi-permeable membrane 3 into a blood chamber 4 and a dialysis fluid chamber 5. The blood chamber is part of the extracorporeal blood circulation (not shown), and the dialysis fluid chamber 5 is part of the dialysis fluid system 1. Central control and computation unit 100 operates and monitors the blood treatment device and the dialysis fluid system. Control and computation unit 110 of the device according to the invention is part of the central control and computation unit 100 of the blood treatment device in the exemplary embodiment. However, the control and computation unit 110 may also be separate from the central control and computation unit 100 and connected to the latter by data lines.

The dialysis fluid system has a mixing chamber 6 for mixing fresh dialysis fluid from clean water and liquid concentrates. The dialysis fluid system has line 7 for carrying clean water, a passive membrane 8 being connected thereto, forming delivery means with a metering function in cooperation with a gear pump 8′ (degassing pump) upstream, referred to hereinafter as delivery means 8. Line 7 opens into the mixing chamber 6. Delivery means 8 is delivery means for clean water.

Another line 9 opens into line 7 downstream from delivery means 8. Delivery means 10 is connected to line 9. Delivery means 10 in the exemplary embodiment is a dosing pump for sodium bicarbonate concentrate. Dosing pump 10 is embodied as a diaphragm pump.

Another line 11 also opens into line 7 at another location which is also downstream from the delivery means 8. Delivery means 12 is connected to line 11. In this exemplary embodiment, the delivery means 11 is a dosing pump for acid concentrate. The dosing pump 12 is designed as a diaphragm pump.

Essentially in the exemplary embodiment for performing the inventive method, the delivery means 8, the delivery means 10 or the delivery means 12 may be used to deliver a liquid into the mixing container. In the exemplary embodiment, performing the inventive method is explained on the example of delivery means 10 as the first delivery means.

In other embodiments, any other pumps arranged upstream from the mixing chamber 6 may be selected as the first delivery means. The choice of the first delivery means may be made automatically by the control and computation unit.

A line 17 leads downstream from the mixing chamber 6 to the dialysis fluid chamber 5 of the dialyzer 2. One delivery means 18 is connected to line 17. In this exemplary embodiment, this delivery means is a gear pump 18, which is part of a balancing device 21. A bypass line 19 having a bypass valve 20 is provided in the parallel connection to the line 17. The bypass valve 20 is closed during operation of the gear pump 18.

In this exemplary embodiment, the inventive method and the inventive device are described in combination with the gear pump 18 as the second delivery means.

In other embodiments, any other pumps arranged downstream from the mixing chamber 6 may be selected as the second delivery means according to the invention. The choice of the second delivery means may be made automatically by the control and computation unit.

The control and computation unit 110 may have means for selecting one of the delivery means (8, 10, 12) for delivering a first delivery volume into the mixing chamber 6. The choice may also be made fixedly in the control and computation unit 110 or may be made by the user through user intervention, for example, via the touchscreen of the blood treatment device (not shown in FIG. 1).

The control and computation unit 110 may also have means for selecting one of the delivery means arranged downstream from the mixing chamber, for example, gear pump 18 or other pumps arranged downstream from the mixing chamber (not shown in FIG. 1) for delivering a second delivery volume out of the mixing chamber 6. The choice may again be fixedly set in the control and computation unit 110 or may be set by the user by user intervention, for example, via the touchscreen of the blood treatment device (not shown in FIG. 1).

In the exemplary embodiment, the diaphragm pump 10 is selected by the control and computation unit 110 as the first delivery means. The gear pump 18 is selected as the second delivery means. A delivery stroke of the diaphragm pump 10 corresponds to the stroke volume delivered in a complete pump stroke. Alternatively, however, the delivery stroke could also include part of the complete pump stroke, for example, when the pump drive is a stepping motor.

The control and computation unit 110 has means for causing the delivery of a predetermined delivery volume of the first delivery means, for example, by stipulating a number of delivery strokes of the diaphragm pump 10. The delivery strokes are prompted by control intervention measures 10a. Furthermore, the control and computation unit 110 has means for ordering the delivery of a predetermined delivery volume of the second delivery means, for example, by stipulating a step angle per delivery step and a number of cuts by means of the control intervention measures 18a on the stepping motor of the gear pump 18.

The inventive device has a pressure sensor 13, which measures the relative pressure in the mixing chamber 6. The measured values of pressure sensor 13 are transmitted to the control and computation unit 110, where they are stored in the data memory 120 for analysis.

In addition, the control and computation unit 110 has a data memory 120.

The pressure differences being sought are calculated with the aid of a computer program using program code to command the machine steps of the method and to analyze the measurement results. The equations for the calculations are implemented in the program code. The program code is stored in the control and computation unit 110.

The computer program may be a computer program product with the program code stored on a machine-readable carrier for commanding the machine steps of the method. The program code is stored in the control and computation unit 110. The control and computation unit 110 has a data memory 120.

The computer program with program code for commanding the machine steps of the method and for analyzing the measurement results starts the calculations as soon as the delivery of the given first and second delivery volumes is concluded.

The total internal volume of the mixing chamber 6 is known and amounts to 350 mL in this exemplary embodiment. The initial liquid volume 16 in the mixing chamber 6 is calculated from the measured value of the filling level measurement device 15. The initial air volume 14 in the mixing chamber is calculated by the control and computation unit 110 as the difference in the total internal volume and the liquid volume and amounts to 242 mL in the exemplary embodiment. The temperature in the mixing chamber is 37° C. and is assumed to be constant while the method according to the invention is being performed.

The number of delivery strokes of the first delivery means to be performed is stipulated in the exemplary embodiment as m=50 in the central control of the blood treatment device. The stroke volume of diaphragm pump 10 in the exemplary embodiment is stipulated as being 1 mL and corresponds to the delivery volume of a single complete delivery stroke of diaphragm pump 10. The control and computation unit 110 starts and stops the delivery strokes of the diaphragm pump through control intervention measures 10a. With each delivery stroke, 1 mL liquid is pumped into mixing chamber 6. There is no return flow of liquid through the diaphragm pump 10. The pressure in the mixing chamber increases with each delivery stroke according to the Boyle-Mariotte law, because the liquid volume increases by the amount of one delivery stroke with each delivery stroke, and as a countermeasure, the air volume decreases by the same amount of the delivery stroke with each delivery stroke. The air is therefore compressed by the same amount with each delivery stroke. This relationship is described by equation (7). The thermodynamic basis for equation (7) is the Boyle-Mariotte law applied to the changes in state of the air volume in the mixing container caused by the delivery strokes.

P₀·V_{vessel,air, 0}=P₁·(V_{vessel,air,0}−m·V_{pump,1}) (7)

The number of delivery steps of the second delivery means to be performed is also predetermined at n=50 in the central control of the blood treatment device in this exemplary embodiment. The delivery volume for delivery step of the gear pump 18 is also 1 mL in this exemplary embodiment and thus corresponds to the delivery volume of a single delivery stroke of the diaphragm pump 10. The control and computation unit 110 stops and starts the delivery steps by controlling the stepping motor of the gear pump 18 with control intervention measures 18a. With each progress of gear pump 18, 1 mL liquid is pumped out of mixing chamber 6. There is no return flow of liquid through the gear pump 18. The pressure in the mixing chamber is reduced according to the Boyle-Mariotte law with each delivery step of the gear pump 18 because the liquid volume in the mixing chamber drops with each delivery step, and as a countermeasure, the air volume is depressurized by the same amount with each delivery step. Therefore, the air is depressurized by the same amount with each delivery step. This relationship is described by equation (8).

P₀·V_{vessel,air,0}=P₂·(V_{vessel,air,0}−m·V_{pump,1}+n·V_{pump,2}) (8)

In equations (7) and (8), P₀ denotes the absolute pressure in the container before delivering the first liquid volume, V_{vessel,air,0} denotes the initial air volume in the container, V_{pump,1} denotes the stroke volume of the first diaphragm pump 10, V_{pump,2} denotes the delivery volume per delivery step of the gear pump 18, P₁ denotes the absolute pressure in the container in the mixing chamber after delivering the first liquid volume, P₂ denotes the absolute pressure in the mixing chamber after delivering the second liquid volume, m denotes the number of pump strokes of the first diaphragm pump 10, and n denotes the number of steps of the gear pump 18, where m may be the same as n.

A first check of the delivery performances with the normal stroke volume of both delivery means 10 and 18 according to the invention yields the plot of the pressure changes in mixing chamber 6 of the dialysis fluid system from FIG. 1, as shown in FIG. 2. FIG. 2 shows that the delivery volume of the gear pump 18 is slightly greater than the delivery volume of the diaphragm pump 10 because the pressure after the second delivery process is smaller than the pressure before the first delivery operation. However, the pressure after the second delivery operation is above the lower pressure limit value and below the upper pressure limit value. It is concluded from this that the volume error is within the tolerance range. The delivery performances of pumps 10 and 18 do not yield any volume error. Since the probability of the simultaneous failure of both pumps 10 and 18 is sufficiently small it is concluded that the delivery performances of both diaphragm pumps 10 and 18 are in order.

A second check of the delivery performances according to the invention with a faulty stroke volume of one of the two pumps 10 and 18 yields the plot of the pressure changes in the mixing chamber of the dialysis fluid system from FIG. 1 as shown in FIG. 3. FIG. 3 shows that the delivery volume of the gear pump 18 is lower than the delivery volume of the diaphragm pump 10 because the pressure is greater after the second delivery operation than the pressure before the first delivery operation. The pressure after the second delivery operation is above the upper pressure limit value. It is concluded from this that the volume error is outside of the tolerance range and at least one of the pumps 10, 18 is defective.

A third check of the delivery performances according to the invention with defective delivery volume of one of the two pumps 10 and 18 yields the plot of the pressure changes in the mixing chamber of the dialysis fluid system from FIG. 1, as shown in FIG. 4. FIG. 4 shows that the delivery volume of the gear pump 18 is greater than the delivery volume of the diaphragm pump 10, because the pressure after the second delivery operation is lower than the pressure before the first delivery operation. The pressure after the second delivery operation is below the lower pressure limit value. It is concluded from this that the volume error is outside of the tolerance range and that at least one of the pumps 10, 18 is defective.

The diaphragm pump 10 is known to be very reliable as a delivery means, whose delivery performance, experience has shown, changes only very slightly in the course of the operating time. Experience has shown that the delivery performance may change to a relevant extent because of the greater wear on the mechanical components in the case of gear pump 18. The delivery performance of the diaphragm pump may therefore be used as a reference for checking the delivery performance of the gear pump 18. In other words, in the present exemplary embodiment, a volume error found in the first and/or second delivery means is attributed exclusively to the gear pump 18 because experience has shown that the probability of a defect in the gear pump 18 is substantially higher than the probability of a defect in the diaphragm pump 10.

In another step, after an inadmissible volume error has been detected by the control and computation unit 110, a control intervention measure 18a is initiated on the pump drive of the gear pump 18, for example, by correcting the step angle, so that the volume error is automatically corrected.

An iterative repetition of the inventive check of the delivery performances with a subsequent correction may also be performed until the volume error has been corrected.

Similarly a cross-comparison of pumps 8, 10 and 12 with pump 18 may be performed, or in other embodiments a cross-comparison of other pumps (not shown in FIG. 1) upstream and downstream from the mixing chamber 6 may also be performed. The inventive method here is implemented as a plausibility check with several pairs of pumps. For example, the delivery volumes of pumps 8 and 18, 10 and 18 as well as 12 and 18 may be compared with one another in three combinations.

If a volume error is detected in the present exemplary embodiment, for example, in a cross-comparison of pumps 10, 12 with gear pump 18 in both combinations, then the volume error is assigned to the gear pump 18 and the volume error is corrected by control intervention measures 18a on the pump drive of the gear pump 18.

However, if a volume error is detected in only one combination in the cross-comparison of pumps 10, 12 with gear pump 18 and is not detected in the other combination, then an error message is output and no further cross-comparison of delivery means is performed because an allocation of the volume error to gear pump 18 is not plausible. Consequently, no control intervention measures are performed. The central control and computation unit in this error case receives an error message from the control and computation unit 110 that an extracorporeal blood treatment must not be performed or continued using defective pumps. In such a case, a traditional check of pump performance by a service technician would be necessary because at least one of the two diaphragm pumps 10, 12 is defective. A warning to request a service technician may be displayed on the display screen of the blood treatment device.

In all embodiments, the start of an extracorporeal blood treatment may be suppressed by the control and computation unit when an inadmissible deviation in the delivery performance of one or more delivery means is ascertained. The safety of the extracorporeal blood treatment can be increased in this way.

According to the invention, the problems formulated are solved by the present invention with the exemplary embodiment presented here. However, the present invention is not limited to this exemplary embodiment.

List of reference numerals

| Reference numeral | Item |
|---|---|
| 1 | dialysis fluid system |
| 2 | Dialyzer |
| 3 | semipermeable membrane |
| 4 | blood chamber |
| 5 | dialysis fluid chamber |
| 6 | mixing chamber |
| 7 | first line |
| 8 | diaphragm chamber |
| 8^{l} | gear pump |
| 8a | control intervention measure |
| 9 | second line |
| 10 | second delivery means, dosing pump/gear pump |
| 10a | control intervention measure |
| 11 | third line |
| 12 | third delivery means, dosing pump/gear pump |
| 12a | control intervention measure |
| 13 | pressure sensor |
| 14 | initial air volume |
| 15 | filling level measurement device |
| 16 | initial fluid volume |
| 17 | line |
| 18 | gear pump |
| 18a | control intervention measure |
| 19 | bypass line |
| 20 | bypass valve |
| 21 | balancing device |
| 100 | central control and computation unit |
| 110 | control and computation unit |
| 120 | data memory |

1. A method for checking the delivery performance of at least one first and/or second delivery means of a device for extracorporeal blood treatment,
comprising the steps:
measuring the pressure in a closed container filled at least partially with air by a first pressure measurement,
delivering a first liquid volume into the container with the first delivery means,
measuring the pressure in the container by a second pressure measurement after delivering the first liquid volume into the container,
delivering a second liquid volume out of the container with the second delivery means,
measuring the pressure in the container by a third pressure measurement after delivering the second liquid volume out of the container,
evaluating at least the measured values of the first pressure measurement and the third pressure measurement as a criterion for the deviation in the delivery performance of the second delivery means from the delivery performance of the first delivery means,
or comprising the steps
measuring the pressure in a closed container filled at least partially with air and at least partially with liquid by a first pressure measurement,
delivering a first liquid volume out of the container with the first delivery means,
measuring the pressure in the container with a second pressure measurement after delivering the first liquid volume out of the container,
delivering a second liquid volume into the container with the second delivery means,
measuring the pressure in the container with a third pressure measurement after delivering the second liquid volume into the container,
evaluating at least the measured values of the first pressure measurement and the third pressure measurement as a criterion for the deviation of the delivery performance of the second delivery means from the delivery performance of the first delivery means.

2. The method according to claim 1, comprising the additional steps:
comparing the measured value of the third pressure measurement with an upper limit value and/or a lower limit value, and
deducing an inadmissible deviation in the delivery performance of the at least one first and/or second delivery means when the value drops below the lower limit value or exceeds the upper limit value.

3. The method according to claim 1 comprising the additional step:
calculating the pressure difference from the measured value of the first pressure measurement and the measured value of the third pressure measurement as a measure of the deviation in the delivery performance of the second delivery means from the delivery performance of the first delivery means.

4. The method according to claim 3 comprising the additional step:
comparing the pressure difference with an upper limit value and/or a lower limit value and deducing an inadmissible deviation in the delivery performance of the first and/or second delivery means when the value drops below the lower limit value or exceeds the upper limit value.

5. The method according to claim 3 comprising the additional step:
calculating a volume difference as the product of the system compliance and the pressure difference from the measured value of the first pressure measurement and the measured value of the third pressure measurement.

6. The method according to claim 5 comprising the additional step:
calculating a volume error from the volume difference minus the target value difference from the target value of the first delivery volume and the target value of the second delivery volume.

7. The method according to claim 6 comprising the additional steps:
comparing the volume error with a predetermined lower limit value for the volume error and/or a predetermined upper limit value for the volume error and deducing an inadmissible deviation in the delivery performance of at least the first and/or second delivery means when the value falls below the lower limit value or exceeds the upper limit value.

8. The method according to claim 2 comprising the additional step:
output of an acoustic and/or optical alarm when the value drops below the lower limit value or exceeds the upper limit value.

9. The method according to claim 2 comprising the additional step:
automatically performing a correction of the delivery performance of at least the first or second delivery means.

10. The method according to claim 1 comprising the additional steps:
predetermining the first delivery volume on a first stepping motor by predetermining at least a first number of steps and a first step angle,
and/or
predetermining the second delivery volume on a second stepping motor by predetermining at least a second number of steps and a second step angle.

11. The method according to claim 1 comprising the additional step:
predetermining the first delivery volume of the first delivery means by predetermining a first delivery time and/or predetermining the second delivery volume of the second delivery means by a second delivery time.

12. A device for checking on the delivery performance of at least one first and/or second delivery means of a device for extracorporeal blood treatment, comprising a closed container filled at least partially with air,
or
a closed container filled at least partially with air and at least partially with liquid,
at least one means for measuring the pressure in the container,
a first delivery means in fluid connection with the container through a first liquid line,
a second delivery means in fluid connection with the container through a second liquid line,
a control and computation unit configured for controlling the first delivery means,
for delivering a first liquid volume into the container filled at least partially with air,
or
delivering a first liquid volume out of the container filled at least partially with air and at least partially with liquid,
controlling the second delivery means, for delivering a second liquid volume out of the container filled by the first delivery means with a first liquid volume,
or
for delivering a second liquid volume into the container emptied of a first liquid volume by the first delivery means,
measuring the pressure in the container with a first pressure measurement before starting the delivery of the first liquid volume,
measuring the pressure in the container with a second pressure measurement after stopping the delivery of the first liquid volume,
measuring the pressure in the container with a third fresher measurement after stopping the delivery of the second liquid volume,
analyzing at least the measured values of the first pressure measurement and the third pressure measurement as a criterion for checking the delivery performance of the first delivery means and/or of the second delivery means.

13. The device according to claim 12, characterized in that the control and computation unit is additionally designed for
comparing the measured value of the third pressure measurement with an upper limit value and/or a lower limit value and deducing an inadmissible deviation in the delivery performance of at least the first and/or second delivery means when the value drops below the lower limit value or exceeds the upper limit value.

14. The device according to claim 12, characterized in that the control and computation unit is additionally designed for
calculating the pressure difference from the measured value of the first pressure measurement and the measured value of the third pressure measurement as a criterion for the deviation of the delivery performance of t he second delivery means from the delivery performance of the first delivery means.

15. The device according to claim 14, characterized in that the control and computation unit is additionally designed for
comparing the pressure difference with an upper limit value and/or a lower limit value and deducing an inadmissible deviation in the delivery performance of the first and/or second delivery means when the value drops below the lower limit value or exceeds the upper limit value.

16. The device according to claim 14, characterized in that the control and computation unit is additionally designed for
calculating a volume difference as the product of the system compliance and the pressure difference from the measured value of the first pressure measurement and the measured value of the third pressure measurement.

17. The device according to claim 16, characterized in that the control and computation unit is additionally designed for
calculating a volume error from the volume difference minus the target value difference from the target value of the first delivery volume and the target value of the second delivery volume.

18. The device according to claim 17, characterized in that the control and computation unit is additionally designed for
comparing the volume error with a predetermined lower limit value for the volume error and/or a predetermined upper limit value for the volume error and deducing an inadmissible deviation in the delivery performance of at least the first and/or second delivery means when the value drops below the lower limit value or exceeds the upper limit value.

19. The device according to claim 13, characterized in that the control and computation unit is additionally designed for
output of an acoustic and/or optical alarm when the value drops below the lower limit value or exceeds the upper limit value.

20. The device according to claim 13, characterized in that the control and computation unit is additionally designed for
performing the correction of the delivery performance of at least the first or second delivery means.

21. The device according to claim 12, characterized in that the control and computation unit is additionally designed for
predetermining the first delivery volume on a first stepping motor by predetermining at least one first number of steps and one first step angle,
and/or
predetermining the second delivery volume on a second stepping motor by predetermining at least one second number of steps and one second step angle.

22. The device according to claim 12, characterized in that the control and computation unit is additionally designed for
predetermining the first delivery volume of the first delivery means by predetermining a first delivery time and/or predetermining the second delivery volume of the second delivery means by a second delivery time.

23. The device according to claim 12, characterized in that the first delivery means is a first displacement pump and the delivery means is a second displacement pump.

24. The device according to claim 23, characterized in that the first displacement pump and/or the second displacement pump is/are selected from the group of diaphragm pump, gear pump and piston pump.

25. The device according to claim 12, characterized in that the container is a mixing chamber in a liquid line of a dialysis fluid system, and the first delivery means is arranged in the liquid line upstream from the mixing chamber and the second delivery means is arranged in the liquid line downstream from the mixing chamber.

26. A blood treatment device comprising:
a dialysis fluid system for supplying a dialysis fluid and a device according to claim 12.

27. A computer program product comprising a program code stored on a machine-readable carrier for storage in the control and computation unit (110) of a device for checking on the delivery performance of at least one first and/or second delivery means of a device for extracorporeal blood treatment, having
a closed container filled at least partially with air,
or
a closed container filled at least partially with air and at least partially with liquid,
at least one means for measuring the pressure in the container,
a first delivery means in fluid connection with the container through a first liquid line,
a second delivery means in fluid connection with the container through a second liquid line,
a control and computation unit configured for controlling the first delivery means,
for delivering a first liquid volume into the container filled at least partially with air,
or
delivering a first liquid volume out of the container filled at least partially with air and at least partially with liquid,
controlling the second delivery means,
for delivering a second liquid volume out of the container filled by the first delivery means with a first liquid volume,
or
for delivering a second liquid volume into the container emptied of a first liquid volume by the first delivery means,
measuring the pressure in the container with a first pressure measurement before starting the delivery of the first liquid volume,
measuring the pressure in the container with a second pressure measurement after stopping the delivery of the first liquid volume,
measuring the pressure in the container with a third fresher measurement after stopping the delivery of the second liquid volume,
analyzing at least the measured values of the first pressure measurement and the third pressure measurement as a criterion for checking the delivery performance of the first delivery means and/or of the second delivery means
for prompting the machine steps of the method according to claim 1 when the program code is run in the control and computation unit (110).

28. The computer program product for storage in the control and computation unit (110) of a device according to claim 12 for checking on the delivery performance of at least one first and/or second delivery means of a device for extracorporeal blood treatment, having
a closed container filled at least partially with air,
or
a closed container filled at least partially with air and at least partially with liquid,
at least one means for measuring the pressure in the container,
a first delivery means in fluid connection with the container through a first liquid line,
a second delivery means in fluid connection with the container through a second liquid line,
a control and computation unit configured for controlling the first delivery means,
for delivering a first liquid volume into the container filled at least partially with air,
or
delivering a first liquid volume out of the container filled at least partially with air and at least partially with liquid,
controlling the second delivery means,
for delivering a second liquid volume out of the container filled by the first delivery means with a first liquid volume,
or
for delivering a second liquid volume into the container emptied of a first liquid volume by the first delivery means,
measuring the pressure in the container with a first pressure measurement before starting the delivery of the first liquid volume,
measuring the pressure in the container with a second pressure measurement after stopping the delivery of the first liquid volume,
measuring the pressure in the container with a third fresher measurement after stopping the delivery of the second liquid volume,
analyzing at least the measured values of the first pressure measurement and the third pressure measurement as a criterion for checking the delivery performance of the first
delivery means and/or of the second delivery means for prompting the machine steps of the method according to claim 1 when the program code is run in the control and computation unit (110).
